(19) United States
(12) Patent Application Publication
(10) Pub. No.: US 2009/0318350 A1
(43) Pub. Date: Dec. 24, 2009

(54) PAN-HER ANTAGONISTS AND METHODS OF USE

(76) Inventors: Philip T. Pienkos, Lakewood, CO (US); Daniel J. Monticello, The Woodlands, TX (US)

Correspondence Address: ELMORE PATENT LAW GROUP, PC, 515 Groton Road, Unit 1R, Westford, MA, 01886, US

(21) Appl. No.: 12/398,281

(22) Filed: Mar. 5, 2009

Related U.S. Application Data
(63) Continuation of application No. 12/376,467.
(60) Provisional application No. 60/818,735, filed on Jul. 6, 2006.

(51) Int. Cl. A61K 38/16; C07K 14/00; A61P 35/00

(52) U.S. Cl. 514/12; 530/324

(57) ABSTRACT

The present invention features human epidermal receptor (HER) antagonists. These antagonists are polypeptide variants of ligands of HER. The HER ligand polypeptide variants of the invention possess Pan-HER antagonistic properties and can inhibit at least one HER-mediated biological activity of one or more HER subtypes, such as inhibition of the receptor's kinase activation activity and subsequently, cell proliferation. Such polypeptide variants, and nucleic acids encoding these polypeptide variants can be used therapeutically in situations in which inhibition of HER activity is indicated.

### RELATED APPLICATIONS

This application is a continuation of U.S. application Ser. No. 12/376,467, filed on Feb. 5, 2009 (371 (c) date not yet assigned), which is a US National stage entry of International Application No. PCT/US2007/072747, which designated the United States and was filed on Jul. 3, 2007, published in English, which claims the benefit of U.S. Provisional Application No. 60/818,735, filed on Jul. 6, 2006. The entire teachings of the above applications are incorporated herein by reference.

### GOVERNMENT SUPPORT

The invention was supported, in whole or in part, by a grant number 2R44CA095930-04 from the National Institutes of Health. The Government has certain rights in the invention.

### FIELD OF THE INVENTION

This invention relates to the design of panoramic ligands and ligand variants that are effective in modulating receptor-mediated pathways, especially those pathways implicated in hyperproliferative conditions such as cancer.

### ABBREVIATIONS

BO, domain binding optimization; DNL, dominant negative ligand; EGF, epidermal growth factor; EGFR, epidermal growth factor receptor; GPCR, G-protein coupled receptor; HER, human epidermal receptor; HER1, human epidermal receptor 1; hGH, human growth hormone; IFN, interferon; IGF, insulin-like growth factor; IR, insulin receptor; NGF, nerve growth factor; Pan-HER antagonist, panoramic human epidermal receptor antagonist; TNF, tumor necrosis factor; VEGF, vascular endothelial growth factor.

### BACKGROUND OF THE INVENTION

Interactions between polypeptide ligands and their cognate receptors are critical for a variety of biological processes including maintenance of cellular and organism homeostasis, development, and tumorigenesis. The cell signaling network created by these ligands and receptor interactions is responsible for relaying a majority of the extracellular, intercellular and intracellular signals—handing off signals from one member of the pathway to the next. Modulation of one member of the pathway can be relayed through the signal transduction pathway, resulting in modulation of activities of other pathway members and modulating outcomes of such signal transduction such as affecting phenotypes and responses of a cell or organism to a signal. Diseases and disorders can, and often do, involve dysregulated signal transduction pathways. A goal of therapeutics is to target such dysregulated pathways to restore more normal regulation in the signal transduction pathway. Many ligands can activate multiple independent pathways and the strength of the activation of different pathways can be modulated by the presence or absence of signals generated by other ligands or receptors.

For example, epidermal growth factor (EGF) is a 53 amino acid cytokine which plays an important role in the growth control of mammalian cells. It is proteolytically cleaved from a large integral membrane protein precursor. The amino acid and nucleotide sequences of human EGF (EGF) are, for example, disclosed In Hollenberg, “Epidermal Growth Factor-Urogastrone, A Polypeptide Acquiring Hormonal States”; eds., Academic Press, Inc., New York (1979), pp. 69-110; or Urdea et al., Proc. Natl. Acad. Sci., USA. 80:7461 (1983). The amino acid sequence of EGF is also disclosed in U.S. Pat. No. 5,102,789 and copending U.S. patent application Ser. No. 10/820,640 both of which are incorporated herein by reference in their entirety.

Human Epidermal Receptors (HER), including epidermal growth factor receptors (EGFR), are well known examples of receptor tyrosine kinases. Interaction of HERs with their cognate ligands, or with structurally related ligands, leads to dimerization and activation of the kinase domain. This initiates a signaling cascade, leading to cell division. Dysregulation of HER signaling, such as the overexpression of the genes coding for HER family members has been implicated in a number of pathologies, especially cancers of the breast, ovary, head and neck.

Molecules that target EGFR (HER1) by inhibiting its kinase activity or by interfering with the binding of EGF to EGFR have been shown to inhibit cell proliferation and have been developed as anticancer therapeutics, for example, Iressa® (gefitinib), a tyrosine kinase inhibitor and Erbitux™ (cetuximab), an EGFR-specific monoclonal antibody. Additional molecules including monoclonal antibodies such as Herceptin target other individual members of the HER family. Recently, several monoclonal antibodies and small molecule kinase inhibitors (e.g. Panituzumab and Lapatnib) have entered development as examples of molecules that target multiple HER receptors.

Although these therapeutics have been shown to be effective in some cases, there is still a need for novel therapies for HER-related pathologies, particularly therapeutic compounds which interfere with the entire HER receptor family in a panoramic fashion.

Current methods of synthesis and expression of polypeptides provide a backdrop for the discovery, investigation and validation of new methods of designing optimized ligands or receptors having panoramic therapeutic properties. These optimized molecules can then be exploited in the areas of drug discovery and medicine, including gene therapy.

### SUMMARY OF THE INVENTION

Accordingly, it is an object herein to provide novel ligands and ligand variants for use, among other things, as therapeutics.

One aspect of the invention relates to a human epidermal receptor (HER) ligand variant designed with EGF as the starting druggable ligand which is then modified to a T1E or WVS background and wherein at least one amino acid corresponding to G18, G39, R41 or L47 of human wild-type epidermal growth factor (EGF) is substituted with a different amino acid. Further provided are such HER ligand variants which are Pan-HER antagonist. The HER ligand variants of the invention have substitutions at, for example amino acid G18, said substitutions being with glutamate (G18E), glutamine (G18Q), lysine (G18K), phenylalanine (G18F), or leucine (G18L).

In another aspect of the invention substitution of the T1E or WVS based HER ligand variant is made at the position corresponding to V35 of wild-type EGF wherein the amino acid V35 is substituted with glutamate (V35E).

In another aspect of the invention substitution of the T1E or WVS based HER ligand variant is made at amino acid G39 and is substituted with glutamate (G39E), glutamine (G39Q), lysine (G39K), aspartic acid (G39D) or isoleucine (G39T), leucine (G39L) or phenylalanine (G39F).

In another aspect of the invention substitution of the T1E or WVS based HER ligand variant is made at amino acid R41 is substituted with aspartate (R41D).

In another aspect of the invention substitution of the T1E or WVS based HER ligand variant is made at amino acid L47 and this residue is substituted with glycine (L47G), apartate (L47D) or arginine (L47R).

As such, the invention provides HER ligand variants of having the following sequences: T1E-G39L, T1E-R41D, T1E-L47G, T1E-R41DL47G, WVS-G39L, WVS-R41D and WVS-L47G, and WVS-R41DL47G.

In another aspect of the invention the ligands are PEGylated at K48.

In addition, the invention provides that these HER ligand variants act as Pan-HER antagonists and this activity is shown to be panoramic against at least two members selected from the group consisting of HER1, HER3 and HER4.

Particularly preferred by the present invention is the HER ligand variant having a WVS background and wherein the amino acid position that corresponds to amino acid L47 of human wild-type epidermal growth factor (EGF) is substituted with another amino acid and wherein the amino acid position that corresponds to amino acid R41 of human wild-type epidermal growth factor (EGF) is substituted with another amino acid.

This preferred ligand variant may also have at least one substituted, modified, swapped, or inverted feature.

Also provided by the present invention are pharmaceutical compositions comprising the HER ligand variants and Pan-HER antagonists described herein alone or in combination with a pharmaceutically acceptable carrier.

In one aspect of the invention is provided methods of treating a patient with a disease characterized by over expression of HER or a HER-mediated pathology comprising, administering to the patient, a therapeutically effective amount of a pharmaceutical composition of the present invention.

Diseases amenable to treatment by the compositions of the present invention include, but are not limited to, cancer and psoriasis.

Further, the present invention contemplates treating cancers selected from the group consisting of gliomas, squamous cell carcinomas, breast carcinomas, melanomas, invasive bladder carcinomas, colorectal carcinomas and esophageal cancers.

### DETAILED DESCRIPTION OF THE INVENTION

A description of embodiments of the invention follows.

The present invention features novel therapeutic HER ligand variants termed Pan-HER antagonists, so named because of their capacity to antagonize signaling mediated by two or more human epidermal receptors (HERs).

As is known in the art, ligands that bind human epidermal receptors (HERs) can be grouped into three categories: 1) Those that bind to HER1 alone (EGF, TGF-a, amphiregulin), 2) those that bind to HER3 and/or HER4 (heregulins and neuregulins) and 3) those that bind to HER-1 and HER-4 (betacellulin, heparin-binding EGF, NRG3, epigen, and epiregulin) (Riese and Stem 1998 Bioessays 20:41). Each human epidermal receptor exists as a monomer in the inactive state. Ligand binding promotes either homodimerization or heterodimerization between the bound receptor and other members of the HER family. The various EGF-like growth factors bind with high affinity to ErbB receptors except for HER2, which has no known ligand and has the constitutive ability to form homodimers and heterodimers. HER2 homodimers have been implicated in tumor cell growth, but also are important for cardiac muscle development and repair. (Dougall et al 1994 Oncogene 9:2109, Hynes and Stem 1994, Biochim Biophys Acta 1198:165, Tzahar and Yarden 1998 Biochim Biophys Acta 1377:M25, Negro et al 2004, Recent Prog Horm Res. 59:1.). HER2 is the preferred heterodimeric partner of the other HER receptors (Tzahar et al 1996, Mol Cell Biol 16:5276, Beerli et al 1995 Mol Cell Biol 15:6496, Karunagaran et al 1996 EMBO J 15:254, Wang et al 1998, PNAS 95:6809). HER3 differs from the other HER family members in that it has a deficient tyrosine kinase domain (Guy et al 1994 PNAS 91:8132) and must associate with another HER-family receptor to trigger signaling.

Examples of HER ligands include mammalian EGF (e.g. human (EGF), pig, cat, dog, mouse, horse and rat). Other examples of HER ligands include transforming growth factor-α (TGFα), betacellulin, heparin-binding EGF-like growth factor (HB-EGF), neuregulins, heregulin (HRG) including HRGα, HRGβ1, HRGβ2 and HRG-factor (NDF), amphiregulin (AR), epigen and epiregulin. The Pan-HER antagonists of the present invention are ligand variants which bind HERs. Preferred HER ligand variants of the invention are based on HER ligands capable of selectively inhibiting HER-mediated biological activity.

According to the present invention the term “Pan-HER antagonist” encompasses any amino-acid based molecule that inhibits, suppresses or causes the cessation of at least one HER-mediated biological activity by reducing, interfering with, blocking, supplanting or otherwise preventing the interaction or binding of a native or active HER ligand to more than one human epidermal receptor (HER) thereby attenuating or inhibiting signaling via a human epidermal receptor).

HERs include HER1, HER2, HER3, and/or HER4 and variant forms of these receptors. It is understood that direct interference with HER1, HER3 and HER4 can provide indirect interference with HER2, by blockading the dimerization partners implicated in much of HER2's role in cancer.

As used herein, the term “antagonist” means any molecule that blocks the ability of a given chemical to bind to its receptor, thereby preventing a biological response. The term antagonist can be used in a functional sense and is not intended to limit the invention to compounds having a particular mechanism of action. For example, the term “antagonist” includes, but is not limited to, molecules that function as competitive antagonists. A “competitive antagonist” is one which binds the receptor but does not trigger the biological activity of the receptor.

“HER-mediated biological activity” as used herein means the intrinsic protein-tyrosine kinase activity of the HER and/or its downstream signal transduction cascade. For example, HER-mediated biological activities include reducing or inhibiting HER kinase activation, signaling, regulation, dimerization, HER-regulated cell proliferation as well as any HER-mediated pathology or phenotypic manifestation evidenced as HER-mediated. The HER ligand variants of the invention are designed to act as Pan-HER antagonists. However, in doing so they may be capable of selectively inhibiting at least one HER-mediated biological activity. Such HER ligand variants, and nucleic acids encoding these variants, can be used therapeutically in situations in which inhibition of HER biological activity is indicated, e.g. cancer, inflammation and the like. As such the present invention encompasses therapeutic Pan-HER antagonists and variants thereof and methods for their design and use in medicine, diagnostics and drug discovery.

### Designing Therapeutic Pan-HER Antagonists and HER Ligand Variants

One aspect of the invention includes the design of HER ligand variants that function as Pan-HER antagonists. This method comprises selecting a druggable ligand and performing domain binding optimization (DBO) on the selected druggable ligand. Optionally, druggable ligands may undergo optimization prior to DBO. Once a druggable ligand has undergone DBO, the ligand can then be assayed for biological activity as a Pan-HER antagonist. Optionally, it may be desired to assay the druggable ligand for biological activity as a Pan-HER antagonist prior to, between or during DBO. Those druggable ligands capable of inhibiting a HER-mediated biological activity as Pan-HER antagonist are identified or termed therapeutic Pan-HER antagonists. The therapeutic Pan-HER antagonists identified by the methods of the present invention are useful in the treatment of diseases or disorders resulting from or characterized by dysregulated HER receptor-mediated cell signaling events and HER-mediated pathologies.

### Selection of a Druggable Ligand

As a starting point, the design method disclosed herein begins with the selection of a druggable ligand. “Druggable ligands” include any ligand which may serve as a starting ligand for the methods of the present invention. These ligands are selected from known receptor ligands or any polypeptide sequence designed to function as a druggable ligand. For example, in copending application U.S. application Ser. No. 11/172,611, filed Jun. 30, 2005, the entire teachings of which are incorporated herein by reference, known HER ligands are used as starting points for investigation.

The present invention contemplates the use and investigation of EGF homologs, analogs and fragments of the EGF. The term “homolog” refers to the corresponding polypeptides of HER ligands from other species having substantial identity to human wild-type HER ligands. These homologs may be modified and optimized according to the present invention to produce Pan-HER antagonists. For example, homologs of EGF polypeptide sequences from various mammalian species are disclosed in Table 1.

TABLE 1
EGF Homologs

| | | SEQ |
|---|---|---|
| | | ID |
| PROTEIN SEQUENCE | Species | No. |
| NSDSECPLSHDGYCLHDGVCMYIEALDKYACNCVVGYIG | Human | 1 |
| ERCQYRDLKWWELR | | |
| NSYSECPPSHDGYCLHGGVCMYIEAVDSYACNCVFGYVG | Pig | 2 |
| ERCQHRDLKWWELR | | |
| NSYQECPPSYDGYCLYNGVCMYIEAVDRYACNCVFGYVG | Cat | 3 |
| ERCQHRDLK-WELR | | |
| NGYRECPSSYDGYCLYNGVCMYIEAVDRYACNCVFGYVG | Dog | 4 |
| ERCQHRDLK-WELR | | |
| NSYPGCPSSYDGYCLNGGVCMHIESLDSYTCNCVIGYSG | Mouse | 5 |
| DRCQTRDLRWWELR | | |
| NSYQECSQSYDGYCLHGGKCVYLVQVDTHACNCVVGYVG | Horse | 6 |
| ERCQHQDLR----- | | |
| NSNTGCPPSYDGYCLNGGVCMYVESVDRYVCNCVIGYIG | Rat | 7 |
| ERCQHRDLRWWKLR | | |

As used herein, the term “analog” refers to compounds whose structure is related to that of another compound but whose chemical and biological properties may be quite different.

In selecting a starting ligand, the known or predicted structure of the selected druggable ligands of the present invention must present, contain or be designed to contain two or more receptor binding surfaces. Structurally, any amino acid-based molecule meeting the criterion defined above is considered a druggable ligand. Receptor binding surfaces may be distinct and separable surfaces, adjacent surfaces or may overlap in space or sequence (i.e., may each utilize the same or common amino acids as a component of the surface).

As the term is used herein, “receptor binding surfaces” are motifs found in druggable ligands and HER ligand variants of the invention which serve as the site of interaction between a ligand and a receptor. The receptor binding surfaces may be defined by a particular amino acid sequence or result from protein folding, e.g., when surfaces are created by nonadjacent amino acids coming into proximity due to electrostatic or thermodynamic energy minimization of the overall sequence of the polypeptide to produce secondary and/or tertiary protein structures.

The corresponding motif in a receptor which serves as the site of interaction between a druggable ligand or HER ligand variant and receptor is herein referred to as the “target receptor domain.”

As used herein the term “ligand” is used to designate an amino acid-based molecule capable of specific binding to a receptor as herein defined. The definition includes any native ligand for a receptor or any region or derivative thereof retaining at least a qualitative receptor binding ability. Specifically excluded from this definition are antibodies to a receptor and noncovalent conjugates of an antibody and an antigen for that antibody.

The terms “native ligand” and “wild-type ligand” are used interchangeably and refer to an amino acid sequence of a ligand occurring in nature (“native sequence ligand”), including mature, pre-pro and pro forms of such ligands, purified from natural source, chemically synthesized or recombinantly produced. Native ligands that can activate receptors are well known in the art or can be prepared by art known methods.

In one embodiment of the invention, the HER ligand variants or Pan-HER antagonists act as dominant negative ligands (DNLs). In this regard, the term “dominant negative” is used to describe that type of ligand, when altered or modified to differ from the native or wild-type ligand in any respect, results in a ligand that retains binding affinity for a wild-type binding partner (e.g., a receptor) but inhibits the function or signaling of the wild-type binding partner.

The present invention contemplates the design of HER ligand variants, Pan-HER antagonists and dominant negative ligands, as that term applies to the aforementioned functional properties, as well as HER ligand variants, Pan-HER antagonists and dominant negative ligands which have as their design reference point, other HER ligand variants, Pan-HER antagonists and dominant negative ligands. These further designed HER ligand variants, Pan-HER antagonists and dominant negative ligands may be the result of further optimization of properties in addition to or beyond binding and signal inhibition. For example, once optimized over a first HER ligand variant, Pan-HER antagonist or dominant negative ligand, a HER ligand variant, Pan-HER antagonist or dominant negative ligand may then be the starting point for further optimization meaning that, in the design scheme, the resultant compound would then become the starting compound. Therefore, a “HER ligand” or “Pan-HER antagonist” can, in certain contexts, be construed as a “HER ligand variant” or “Pan-HER antagonist variant”, respectively, and vice versa. Furthermore, when used as a starting or reference point for design, a HER ligand variant, Pan-HER antagonist or dominant negative ligand may also be referred to or considered a druggable ligand.

As used herein the term “dominant negative ligand activity” refers to the functions associated with dominant negative ligands (e.g., binding a receptor but inhibiting a function of the receptor).

The druggable ligands, HER ligand variants, and Pan-HER antagonists of the present invention are amino acid-based molecules. These molecules may be “peptides,” “polypeptides,” or “proteins.” While it is known in the art that these terms imply relative size, these terms as used herein should not be considered limiting with respect to the size of the various amino acid-based molecules referred to herein and which are encompassed within this invention. Thus, any amino acid sequence comprising at least one of the HER ligand variants, Pan-HER antagonists or their receptor binding surfaces disclosed herein, and which binds to any receptor is within the scope of this invention.

The terms “amino acid” and “amino acids” refer to all naturally occurring L-alpha-amino acids. The amino acids are identified by either the one-letter or three-letter designations as listed in Table 2.

TABLE 2
Naturallly occurring amino acids

| Three letter | One letter | Amino acid |
|---|---|---|
| Asp | D | aspartic acid |
| Ile | I | isoleucine |
| Thr | T | threonine |
| Leu | L | Leucine |
| Ser | S | Serine |
| Tyr | Y | Tyrosine |
| Glu | E | Glutamic acid |
| Phe | F | phenylalanine |
| Pro | P | Praline |
| His | H | Histidine |
| Gly | G | Glycine |
| Lys | K | Lysine |
| Ala | A | Alanine |
| Arg | R | Arginine |
| Cys | C | Cysteine |
| Trp | W | tryptophan |
| Val | V | Valine |
| Gln | Q | glutamine |
| Met | M | methionine |
| Asn | N | asparagine |

The amino acid sequences of the HER ligand variants and Pan-HER antagonists of the invention may comprise naturally occurring amino acids and as such may be considered to be proteins, peptides, polypeptides, or fragments thereof. Alternatively, the HER ligand variants and Pan-HER antagonists may comprise non-naturally occurring amino acids or both naturally and non-naturally occurring amino acids.

The term “amino acid sequence variant” refers to molecules with some differences in their amino acid sequences as compared to a native sequence. The amino acid sequence variants may possess substitutions, deletions, and/or insertions at certain positions within the amino acid sequence of a native ligand.

Ordinarily, variants will possess at least about 70% homology to a native ligand, and preferably, they will be at least about 80%, more preferably at least about 90% homologous to a native ligand.

“Homology” as it applies to amino acid sequences is defined as the percentage of residues in the candidate amino acid sequence that are identical with the residues in the amino acid sequence of a native ligand after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” is meant the corresponding polypeptides of HER ligands from other species having substantial identity to human wild-type HER ligands. As used herein, the term “analog” refers to compounds whose structure is related to that of another compound but whose chemical and biological properties may be quite different. In reference to the HER ligand variants and Pan-HER antagonists of the present invention, an analog includes polypeptide variants which differ by one or more amino acid alterations, e.g., substitutions, additions or deletions of amino acid residues that still maintain the functional properties (e.g., dominant negative function, inhibition, attenuation, etc.) of the parent polypeptide. As stated above, parent molecules (i.e., the reference point for comparison) may comprise druggable ligands, HER ligand variants, Pan-HER antagonists, DNLs or variants thereof.

As described herein, the HER ligand variants and Pan-HER antagonists produced by the methods of the present invention, their homolog variants and analogs may have substantial sequence identity to wild-type ligands. However, it is appreciated that substantial sequence identity is not a single defining feature of the compounds of the present invention. Structural components are also factors when considering identity of a variant to the parent molecule.

As used herein, “substantial sequence identity” means at least 60% sequence identity, preferably at least 70% identity, preferably at least 80% and more preferably at least 90% sequence identity to the amino acid sequence of starting ligand (or domains thereof in the instance where the variant is a chimera produced by swapping domains), while maintaining HER-mediated biological activity. In other embodiments, the HER ligand variants and Pan-HER antagonists of the present invention have at least 91%, at least 92%, at least 93%, at least 94%, at least 95% at least 96%, at least 97%, or at least 98% sequence identity to the amino acid sequence of wild-type human ligand, while maintaining HER-mediated biological activity.

The percent identity of two amino acid sequences can be determined by aligning the sequences for optimal comparison purposes (e.g., gaps can be introduced in the sequence of a first sequence). The amino acids at corresponding positions are then compared, and the percent identity between the two sequences is a function of the number of identical positions shared by the sequences (i.e., % identity=# of identical positions/total # of positions×100). The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the BLASTN and BLASTX programs (version 2.2) as described in Schaffer et al., Nucleic Acids Res., 29:2994-3005 (2001).

The term “derivative” is used synonymously with the term “variant” and refers to a molecule that has been modified or changed in any way relative to a reference molecule or starting molecule. As used herein derivative and variant HER ligands or Pan-HER antagonists are amino acid-based molecules which are modified, altered, improved or optimized relative to a starting parent molecule.

The present invention contemplates several types of HER ligand and Pan-HER antagonist variants and derivatives. These modifications can be useful to alter receptor specificity (either broaden specificity such that the variant binds to additional receptors or target specificity towards a specific receptor or narrow specificity to less than all of the family of receptors but not less than two) or to alter phenotypic outcomes. Also included in the modifications of the compounds of the invention are domain swapping and/or domain modification strategies as discussed herein.

As such, included within the scope of this invention are amino acid-based molecules containing substitutions, insertions and/or additions, deletions and covalently modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to provide sites for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In one aspect, the present invention features a HER ligand variant or Pan-HER antagonist designed from a naturally occurring HER ligand that has at least one amino acid substitution at amino acid position that corresponds to any one or more amino acids selected from Gly 18 (G18), Val 35 (V35), Gly 39 (G39), Arg 41 (R41) and Leu47 (L47) of wild type EGF.

As used herein, the phrase “amino acid position that corresponds to” means that when the starting HER ligand is aligned with the variant for optimal comparison, the amino acids that appear at or near the positions identified may be substituted with another amino acid. See U.S. Pat. No. 7,470,769 B2 issued on Dec. 30, 2008, incorporated herein by reference.

According to the embodiments of the invention, G18 is replaced by glutamate (G18E), glutamine (G18Q), lysine (G18K), phenylalanine (G18F), or leucine (G18L). In one embodiment, G18 is replaced by phenylalanine (G18F) or leucine (G18L). In yet another embodiment, G18 is replaced by phenylalanine (G18F). Additionally or alternatively, G39 is replaced by glutamate (G39E), glutamine (G39Q), lysine (G39K), aspartic acid (G39D) or isoleucine (G39I), or leucine (G39L). In another embodiment G39 is replaced by phenylalanine (G39F), leucine (G39L), aspartic acid (G39D), or isoleucine (G39I). G39L is preferred. Additionally or alternatively, R41 is replaced by aspartate (R41D), alanine (R41A), leucine (R41L), tyrosine (R41Y), glutamine (R41Q), glutamate (R41E), or lysine (R41K). Additionally or alternatively, L47 is replaced by Glycine (L47G), aspartate (L47D) or arginine (L47R).

Modifications at G18, G39, R41 and L47 in wild type are believed to be responsible for preventing, banishing or abrogating binding of the HER variant to HER1 Domain III. In other words, the variant is believed to not bind to Domain III of HER1.

Additionally or alternatively, V35 is replaced by glutamate (V35E). It is believed that the modification to V35 is responsible for improved binding of the variant to Domain I of HER1. In combination, then, mutations at V35 along with mutations at G18 and/or G39 and/or R41 and/or L47 result in a polypeptide with antagonist properties wherein the ligand is characterized by a disrupted binding at Domain III but stronger binding at Domain I.

“Insertional variants” are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” are those with one or more amino acids in the native or starting amino acid sequence removed. Ordinarily, deletional variants will have one or more amino acids deleted in a particular region of the molecule.

“Covalent derivatives” include modifications of a native or starting ligand with an organic proteinaceous or non-proteinaceous derivatizing agent, and post-translational modifications. Covalent modifications are traditionally introduced by reacting targeted amino acid residues of the ligand with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-ligand antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the ligands used in accordance with the present invention.

Other post-translational modifications include hydroxylation of proline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which ligands of the invention are covalently bonded to a nonproteinaceous polymer. The nonproteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The ligands may be linked to various nonproteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

Post-translational variants also include glycosylation variants. The term “glycosylation variant” is used to refer to a ligand having a glycosylation profile different from that of a native or starting ligand. Any difference in the location and/or nature of the carbohydrate moieties present in a HER ligand variant or Pan-HER antagonist as compared to its native or starting counterpart is within the scope herein.

The glycosylation pattern of native or starting ligands can be determined by well known techniques of analytical chemistry, including chromatography (Hardy, M. R. et al., Anal. Biochem., 170, 54-62 (1988)), methylation analysis to determine glycosyl-linkage composition (Lindberg, B., Meth. Enzymol. 28. 178-195 (1972); Waeghe, T. J. et al., Carbohydr. Res. 123, 281-304 (1983)), NMR spectroscopy, mass spectrometry, etc. For ease, changes in the glycosylation pattern of a native or starting ligand are usually made at the DNA level, essentially using the techniques known in the art with respect to the amino acid sequence variants.

Carbohydrate moieties present on a ligand may also be removed chemically or enzymatically. Chemical or enzymatic coupling of glycosydes to the ligands of the present invention may also be used to modify or increase the number or profile of carbohydrate substituents. These methods are described in WO 87/05330 (published 11 Sep. 1987), and in Aplin and Wriston, CRC Crit. Rev, Biochem., pp. 259-306.

Glycosylation variants of the ligands herein can also be produced by exploiting in vivo methods such as the normal processes of an appropriate host cell. Yeast, for example, introduce glycosylation which varies significantly from that of mammalian systems. Similarly, cells having a different species (e.g. hamster, murine, insect, porcine, bovine or ovine) or tissue (e.g. lung, liver, lymphoid, mesenchymal or epidermal) origin than the source of the ligand, are routinely screened for the ability to introduce variant glycosylation.

Amino acid sequences of the druggable ligands, HER ligand variants and Pan-HER antagonists of the invention may be obtained through various means such as chemical synthesis, phage display, cleavage of proteins or polypeptides into fragments, or by any means which amino acid sequences of sufficient length to possess selected properties may be made or obtained.

In one embodiment, the HER ligand variants and Pan-HER antagonists of the invention are produced by expression in a suitable host of a gene coding for the relevant HER ligand variant or Pan-HER antagonist. Such a gene is most readily prepared by site-directed mutagenesis of the wild-type gene, a technique well known in the art.

As such, the present invention also provides nucleic acid molecules encoding a HER ligand variant or Pan-HER antagonist of the invention. The nucleic acid molecules of the present invention can be RNA, for example, mRNA, or DNA. DNA molecules can be double-stranded or single-stranded. The nucleic acid molecule can also be fused to a marker sequence, for example, a sequence that encodes a polypeptide to assist in isolation or purification of the polypeptide. Such sequences include, but are not limited to, those that encode a glutathione-S-transferase (GST) fusion protein, those that encode a hemagglutinin A (HA) polypeptide marker from influenza, and sequences encoding a His tag.

It will be appreciated by those skilled in the art that the design of the expression vector can depend on such factors as the choice of the host cell to be transformed and the level of expression of HER ligand variant or Pan-HER antagonist desired. The expression vectors of the invention can be introduced into host cells to thereby produce the modified polypeptides of the invention, including fusion polypeptides, encoded by nucleic acid molecules as described herein. Molecular biology techniques for carrying out recombinant production of the modified polypeptides of the invention are well known in the art and are described for example, in, Sambrook, et al., Molecular Cloning: A Laboratory Manual (Cold Spring Harbor Lab Press; 3^{rd} ed., 2000).

Alternatively, the HER ligand variant or Pan-HER antagonist of the invention may be produced in whole or in part by chemical synthetic techniques such as by a Merrifield-type synthesis (J. Am. Chem. Soc. 85:2149 (1963), although other equivalent chemical syntheses known in the art may be used. Solid-phase synthesis is initiated from the C-terminus of the peptide by coupling a protected alpha-amino acid to a suitable resin. The amino acids are coupled the peptide chain using techniques well known in the art for the formation of peptide bonds. Chemical synthesis of all or a portion of a HER ligand variant or Pan-HER antagonist of the invention may be particularly desirable in the case of the use of a non-naturally occurring amino acid substituent in the HER ligand variant or Pan-HER antagonist.

### Modifications and Manipulations

In order to design effective therapeutic Pan-HER antagonists according to the methods of the invention, it is necessary to optimize the druggable ligands (i.e., the starting ligand or HER ligand variants) selected. This optimization may include modifications prior to domain binding optimization or afterwards. The process of optimizing may be iterative, requiring several rounds of modifications to optimize each of a number of properties of the druggable ligand or it may occur step-wise in a sequential manner. Modifications may be made singly, or combinatorially to improve or alter one or more properties of the molecules.

In one embodiment of the invention are provided compounds and compositions designed by making modifications to one or more features of the druggable ligands to alter one or more properties of the druggable ligands, said properties selected from the group consisting of optimal pH or pH-activity, digestibility, antigenicity, half-life, bioavailability, the amphipathic properties, ligand-receptor interactions, thermal or kinetic stability, solubility, folding, posttranslational modification, hydrophobicity, hydrophilicity, and any combination thereof. It will be understood by those of skill in the art that the properties listed represent considerations in developing therapeutics, diagnostics and research tools and that other properties of molecules may also need to be considered and optimized depending on the particular application.

As used herein the term “optimized or optimization” refers to the modification or alteration of a molecule such that one or more characteristics of the molecule are improved for a particular purpose as compared to a starting molecule. “Modification” is the result of modifying wherein the thing being modified is changed in form or character. The molecules of the present invention being optimized via modifications include druggable ligands, HER ligand variants and Pan-HER antagonists. For the purposes of the instant invention, these molecules are being optimized for the purpose of creating therapeutic, diagnostic or research reagents.

The modifications of the present invention are herein made to one or more features of the druggable ligands, HER ligand variants or Pan-HER antagonists. “Features” are defined as distinct amino acid sequence-based components of a molecule. Features of the druggable ligands, HER ligand variants or Pan-HER antagonists of the present invention include surface manifestations, local conformational shape, folds, loops, half-loops, domains, half-domains, sites, termini or any combination thereof.

As used herein the term “surface manifestation” refers to an amino acid-based component of a druggable ligand, HER ligand variant or Pan-HER antagonist appearing on an outermost surface.

As used herein the term “local conformational shape” means an amino acid-based structural manifestation of a druggable ligand, HER ligand variant or Pan-HER antagonist which is located within a definable space of the druggable ligand, HER ligand variant or Pan-HER antagonist.

As used herein the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol, 266 (4): 814-830; 1997).

As used herein the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein the term “domain” refers to a motif of a polypeptide having one or more identifiable structural or functional characteristics or properties (e.g., binding capacity, serving as a site for protein-protein interactions.

As used herein the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein the terms “site” is used synonymous with “amino acid residue” and “amino acid side chain”. A site represents a position within a peptide or polypeptide that may be modified, manipulated, altered, derivatized or varied within the amino acid-based molecules of the present invention.

As used herein the terms “termini or terminus” refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Druggable ligands are in some cases made up of multiple polypeptide chains brought together by disulfide bonds or by non-covalent forces (multimers, oligomers). These sorts of ligands will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, substituting, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Domain Binding Optimization (DBO)

Once a druggable ligand has been selected, and optionally modified or optimized, domain binding optimization (DBO) of the druggable ligand is performed.

As used herein “domain binding optimization” involves making one or more modifications or manipulations as described above to one or more features at a first receptor binding surface of the druggable ligand to disrupt binding of the druggable ligand to a first target receptor domain, and making one or more modifications to one or more features at a second receptor binding surface of the druggable ligand to enhance binding of the druggable ligand to a second target receptor domain. The invention features HER ligand variants and Pan-HER antagonists wherein the binding affinity of the variant or antagonist to one of Domain I or Domain III to an HER is disrupted while maintaining or increasing the binding of the variant to the other of Domain I or III.

In a preferred embodiment, the binding to Domain III of an HER is disrupted and the binding to Domain I to an HER is enhanced.

As stated above, a “target receptor domain” is the corresponding motif in a receptor which serves as the site of interaction between a druggable ligand and receptor.

As used herein the terms “receptor” and “target receptor” may be used interchangeably and refer to the member of the ligand-receptor binding pair which effects alteration of downstream signaling events.

For the purpose of the present invention the receptor is a human epidermal receptor (HER).

HER receptors are present in the cell in a membrane-bound form. They can be found in the cell membrane or in the membranes of endosomes or lysosomes.

Receptor binding surfaces in ligands and target receptor domains in receptors can be determined by methods known in the art, including computational analysis (e.g., molecular modeling), X-ray studies, mutational analyses, antibody binding studies, and random peptide library panning and binding studies. The mutational approaches include the techniques of site-directed mutagenesis, random saturation mutagenesis coupled with selection of escape mutants, insertional mutagenesis, and homolog-scanning mutagenesis (replacement of sequences from human ligands, which bind the corresponding receptor, with unconserved sequences of a corresponding ligand from another animal species, e.g. mouse, which do not bind the human receptor).

In one embodiment of the invention said first and said second target receptor domains are located in the same HER. However the target receptor domains may be located in separate molecules of the same receptor type or in two separate types of receptor molecules. Furthermore, for the purposes of the binding assays, the entire receptor need not be used and binding need only be evaluated using a molecule comprising the target receptor domain. As such, in one embodiment of the invention are methods wherein the disruption or enhancement of binding of the HER ligand variant or Pan-HER antagonist to a said first or a said second target receptor domain is determined by measuring the binding affinity of the HER ligand variant or Pan-HER antagonist to one or more molecules selected from the group consisting of native target HERs containing the target receptor domain, isolated target HER domains and representative target HER moieties.

In one embodiment, the HER ligand variant having at least one amino acid substitution at amino acid position that corresponds to any one or more amino acids selected from Gly 18 (G18), Val 35 (V35), Gly 39 (G39), Arg 41 (R41) and Leu47 (L47) of wild type further comprises at least one feature from a different HER ligand (e.g., a substituted domain, loop, terminus, turn or bend). The variants so created may be further modified in the feature from the different HER ligand or in other ways. These further modifications may be structural or functional as described herein.

In a preferred embodiment, the further feature is the amino terminus of EGF which is believed to enhance the binding affinity of the variant to Domain I of the target HER receptor.

One example of a known EGF variant comprising a substituted amino terminal is the synthetic heregulin (HRG)/EGF chimera known as BiRegulin. BiRegulin (BiR) is a chimeric EGF homolog, in which the amino terminal residues (NSDSE) of EGF, have been replaced with the corresponding residues of HRGβ1 (SHLVK).

Another example of a known EGF variant comprising a substituted amino terminal domain is the EGF/TGFα chimera known as T1E (Stortelers et al., Biochemistry, 2002, 41, 4292-4301). T1E is the result of the introduction of the N-terminal linear region of TGF-α into EGF. Thus the EGF amino terminal residues (NSDSE) are replaced with seven residues from TGF-α (VVSHFND).

Other known EGF variants comprising a modified EGF N-terminus include EGFwvs and EGFwrs. EGFwvs and EGFwrs are the result of testing for enhanced affinity to EGFR by screening random mutations of amino terminus residues 2, 3, and 4 of wild-type EGF (Stortelers et al., Biochemistry, 2002, 41, 8732-8741). These variants of EGF also show high binding affinity, particularly for Her-3. EGFwvs is a variant wherein residues 2 and 3 of EGF are replaced with W and V respectively resulting in a modified N-terminus of EGF having the sequence NWVSE. EGFwrs is a variant wherein residues 2 and 3 of EGF are replaced with W and R respectively resulting in a modified N-terminus of EGF having the sequence NWRSE.

In one embodiment of the invention, certain features or subfeatures are modified, substituted, moved, swapped, inverted, deleted, randomized or duplicated.

For example the B-loop of EGF that corresponds to amino acids 21-30 of mature EGF may be substituted with a corresponding B-loop of another, different, preferably human, HER ligand, such as human transforming growth factor-α (TGFα), betacellulin, heparin-binding EGF-like growth factor (HB-EGF), neuregulins, heregulin (HRG), amphiregulin (AR), epigen and epiregulin. The corresponding B-loop can be incorporated to alter the HER specificity of the ligand or antagonist. For example, if EGF comprising a Leucine to Glycine at position 47 mutation (L47G) is the starting point for designing a HER ligand variant of the invention, the B-loop of L47G is substituted with the corresponding B-loop of a different HER ligand that is known to interact with the specific HER molecule that is intended to be antagonized. Such modified ligands can possess the antagonist activities of the L47G variant with altered binding specificity based on the source of the B-loop swap.

Therefore, to design a Pan-HER antagonist or HER ligand variant of the invention with enhanced targeting of HER1, the EGF B-loop may be substituted with the corresponding B-loop from TGF-alpha, or amphiregulin. To design a HER ligand variant of the invention with enhanced targeting of HER3 and/or HER4, the EGF B-loop may be substituted with the corresponding loop from the neuregulins. HER ligand variants with enhanced targeting of HER1 and HER4, may substitute the EGF B-loop with the corresponding loop from betacellulin, HB-EGF, NRG3 or epiregulin. As HER2 has no known ligand, it can be indirectly targeted for downregulation by blockading the HER1, HER2, or HER4 dimerization partners required for activity, that is, by antagonizing HER1, HER 3 and HER4 using the variants of the invention.

In one embodiment HER ligand variants comprise a substituted feature from another HER ligand including a EGF/HRG chimera, BiRegulin, or the EGF/TGF-α chimera, T1E, in combination with at least one or more amino acid substitutions at positions that corresponds to amino acid Gly 18 (G18) and/or amino acid Gly 39 (G39) and/or amino acid Arg 41 (R41) and/or amino acid Val 35 (V35) and/or amino acid Leu47 (L47) of wild type human EGF.

In one embodiment of the invention, the HER ligand variant comprises BiRegulin (BiR) having at least one amino acid substitution at the leucine of position 47 corresponding to wild type EGF, wherein L47 has been replaced by glycine resulting in the polypeptide variant of the invention designated (BiR-L47G).

In one embodiment of the invention, HER ligand variants of the invention comprising BiRegulin include, but are not limited to, BiR-R41DL47G and BiR-G39L147G.

In one embodiment of the invention, the HER ligand variant comprises T1E having at least one amino acid substitution at the arginine of position 41 corresponding to wild type EGF, wherein R41 has been replaced by aspartate resulting in the polypeptide variant of the invention designated T1E-R41D. Other HER ligand variants of the invention include but are not limited to T1E-R41 DL47G and T1E-L47G.

In one embodiment of the invention, lysine at position 28 (K28) is modified to any of the naturally occurring amino acids. Preferably this modification comprises K28R. Further, variants containing a K28 modification may further be derivatized at lysine 48 with a non-amino acid moiety, such as polyethelyene glycol (PEG).

In another preferred embodiment of the invention, HER ligand variants comprise a variant having a modified feature, such as, for example, a modified amino-terminal EGF domain in combination with at least one or more amino acid substitutions at positions that corresponds to amino acid Gly18 (G18) and/or amino acid Gly 39 (G39) and/or amino acid Arg 41 (R41) and/or amino acid Val 35 (V35) and/or amino acid Leu47 (L47) of wild type human EGF.

In one embodiment of the invention, the HER ligand variant comprises EGFwvs having at least one amino acid substitution at the arginine position 41 corresponding to wild type EGF, wherein the arginine of position 41 corresponding to wild type EGF R41 has been replaced by aspartate and the resulting ligand variant of the invention is designated wvs-R41D.

In one embodiment of the invention, the HER ligand variant comprises EGFwvs having at least one amino acid substitution at the leucine position 47 corresponding to wild type EGF, wherein the leucine of position 47 corresponding to wild type EGF L47 has been replaced by glycine and the resulting polypeptide variant is designated wvs-L47G.

In yet another preferred embodiment, the HER ligand variant comprises EGFwvs having amino acids substitutions at both R41 and L47 resulting in the polypeptide variant of the invention designated wvs-R41DL47G.

The advantage of the HER ligand variants of the invention such as T1E-R41D, wvs-R41DL47G and wvs-L47G is that they are specific for multiple HER targets such as HER1 and HER3 and can antagonize each of these HER subtypes.

It is not necessary that the HER ligand variant of the invention target HER2, because the targeting of HER1, HER3 and HER4 by the variants of the invention effectively blockades all HER2 heterodimerization partners thereby suppressing HER2 biological activity that may be undesirable. It is advantageous to avoid suppression of HER2 homodimerization because such activity has been shown to cause cardiomyopathy, a life threatening side effect.

In one embodiment of the invention the target receptor is selected from the group consisting of HER receptors.

### Binding Studies

As domain binding optimization involves modification of the binding properties of the druggable ligands, it is necessary to perform certain binding assays to assess the resultant binding properties of the ligand after DBO. It is understood that many binding assays for assessing protein-protein binding and ligand-receptor binding are known in the art and within the ability of one of ordinary skill in the art.

The HER ligand variants and Pan-HER antagonists provided by this invention should have an affinity for an HER sufficient to provide adequate binding for the intended purpose. Thus, for use as a therapeutic, the peptide, polypeptide, or protein provided by this invention should have an affinity (Kd) of between about 1-1000 nM for the target receptor. More preferably the affinity is 10 nM. Most preferably, the affinity is 1 nM. For use as a reagent in a competitive binding assay to identify other ligands, the amino acid sequence preferably has affinity for receptor higher than or equal to the authentic ligand.

As used herein the term “binding” includes the formation of one or more ionic, covalent, hydrophobic, electrostatic, or hydrogen bonds between a receptor binding surface of the HER ligand variants and Pan-HER antagonists of the invention and one or more amino acids of a target receptor domain of a target receptor. Binding can be considered “tight” if the HER ligand variant or Pan-HER antagonist is not substantially displaced in an in vitro assay. The HER ligand variant or Pan-HER antagonist is not substantially displaced if at least 50%, preferably at least 70%, more preferably at least about 90%, such as 100%, of the HER ligand variant or Pan-HER antagonist remains bound to a receptor or receptor moiety when competitively challenged with a native ligand. Binding can also be considered tight if the HER ligand variant or Pan-HER antagonist substantially displaces the native ligand from the receptor. The HER ligand variant or Pan-HER antagonist substantially displaces the native ligand if at least 50%, preferably at least 70%, more preferably at least about 90%, such as 100%, of the native ligand is displaced from the receptor.

The binding or bioactive activity of a HER ligand variant or Pan-HER antagonist of the invention can further be assessed by any other suitable assay or other method, wherein the results or activity of such assay are compared to the binding or receptor activity from an assay which measures the binding or receptor activity of wild-type human ligands and receptors.

In one embodiment of the invention, binding studies are performed on libraries of compounds of the invention. Methods of library production can also be used to create the druggable ligand starting molecules of the invention.

In one embodiment of the invention, the modifications made to the druggable ligands or HER ligand variants or Pan-HER antagonists result in or from the production of a library of modified polypeptides. The library of modified polypeptides may comprise a phage library or any other selection or grouping of polypeptide sequences independent of the manner in which they were generated.

As used herein, the term “library” means a collection of molecules. A library can contain a few or a large number of different molecules, varying from about two to about 10¹⁵ molecules or more. The chemical structure of the molecules of a library can be related to each other or be diverse. If desired, the molecules constituting the library can be linked to a common or unique tag, which can facilitate recovery and/or identification of the molecule.

### Phage Panning

Methods for preparing libraries containing diverse populations of various types of molecules such as peptides, proteins, peptoids and peptidomimetics are well known in the art and various libraries are commercially available (see, for example, Ecker and Crooke, Biotechnology, 13:351-360 (1995), and Blondelle et al., Trends Anal. Chem., 14:83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff, John Wiley & Sons 1995), pages 803-861, and Gordon et al., J. Med. Chem., 37:1385-1401 (1994), each of which is incorporated herein by reference). Where a molecule is a peptide, protein or fragment thereof, the molecule can be produced in vitro directly or can be expressed from a nucleic acid, which can be produced in vitro. Methods of synthetic peptide and nucleic acid chemistry are well known in the art.

In addition, a library of molecules can be a library of nucleic acid molecules, which can be DNA, RNA or analogs thereof. For example, a cDNA library can be constructed from mRNA collected from a cell, tissue, organ or organism of interest, or by collecting genomic DNA, which can be treated to produce appropriately sized fragments using restriction endonucleases or methods that randomly fragment genomic DNA. A library comprising RNA molecules also can be constructed by collecting RNA from cells or by synthesizing the RNA molecules chemically. Methods for producing such libraries are well known in the art (see, for example, Sambrook et al., Molecular Cloning: A laboratory manual (Cold Spring Harbor Laboratory Press 1989), which is incorporated herein by reference). Diverse libraries of nucleic acid molecules can be made using solid phase synthesis, which facilitates the production of randomized regions in the molecules. If desired, the randomization can be biased to produce a library of nucleic acid molecules containing particular percentages of one or more nucleotides at a position in the molecule (U.S. Pat. No. 5,270,163, issued Dec. 14, 1993, which is incorporated herein by reference).

In one embodiment of the invention, binding of ligands and receptors is determined using phage panning of a library of ligands. For example, an assay may be performed screening a HER ligand variant or Pan-HER antagonist library which was produced via phage expression.

The screening of very large protein libraries has been accomplished by a variety of techniques that rely on the display of proteins on the surface of viruses or cells. The underlying premise of display technologies is that proteins engineered to be anchored on the external surface of biological particles (i.e., cells or viruses) are directly accessible for binding to ligands without the need for lysing the cells. Viruses or cells displaying proteins with affinity for a ligand can be isolated in a variety of ways including sequential adsorption/desorption form immobilized ligand, by magnetic separations or by flow cytometry (Ladner et al. 1993, U.S. Pat. No. 5,223,409, Ladner et al. 1998, U.S. Pat. No. 5,837,500, Georgiou et al. 1997, Shusta et al. 1999).

The most widely used display technology for protein library screening applications is phage display. Phage display is a well-established and powerful technique for the discovery of proteins that bind to specific ligands and for the engineering of binding affinity and specificity (Rodi and Malowski, Curr. Opin. Biotechnol., 10:87-93; 1999; Wilson and Finlay, Canadian Journal of Microbiology, 44:313-329; 1998). In phage display, a gene of interest is fused in-frame to phage genes encoding surface-exposed proteins, most commonly pIII. The gene fusions are translated into chimeric proteins in which the two domains fold independently. Phage displaying a protein with binding affinity for a ligand can be readily enriched by selective adsorption onto immobilized ligand, a process known as “panning”. The bound phage is desorbed from the surface, usually by acid elution, and amplified through infection of E. coli cells. Usually, 3-6 rounds of panning and amplification are sufficient to select for phage displaying specific polypeptides, even from very large libraries with diversities up to 10¹⁵. Each round of panning enriches the pool of clones in favor of the tightest-binding ligands. Because each phage particle contains both the displayed peptide and the DNA encoding it, the selected peptides can be readily identified by DNA sequencing. Several variations of phage display for the rapid enrichment of clones displaying tightly binding polypeptides have been developed (Duenas and Borrebaeck, 1994; Malmborg et al., 1996; Kjaer et al., 1998; Burioni et al., 1998; Levitan, 1998; Mutuberria et al., 1999; Johns et al., 2000).

The phage panning methods of the present invention involve introduction of an oligonucleotide encoding the HER ligand variants or Pan-HER antagonists of the present invention for expression on the phage particle surface and panning the phage particles against the target receptors or receptor moieties. Phage panning may be used in conjuction with other binding assays such as enzyme linked immunosorbent assay (ELISA) methods.

The methods of the present invention further contemplate the step of repeating the phage panning of the HER ligand variants or Pan-HER antagonists. This repetition may be performed to optimize any or all of the properties of the HER ligand variant or Pan-HER antagonist being investigated. It may also be performed in order to increase the population of domain binding optimized HER ligand variants or Pan-HER antagonists.

### Rational Redesign

In one embodiment of the invention the methods may further comprise the step of rational redesign wherein the steps of selecting druggable ligands and the modifications made in the DBO step to the selected druggable ligands are performed iteratively, either alone or in combination.

For example, within the feature (e.g., B-loop) itself, random mutation or mutation by rational design is shown to further enhance ligand variant binding, particularly to Domain I. The first half of the B-loop, amino acid residues 21-25, and the second half of the B-loop residues 26-30 have been rationally redesigned herein.

In addition, it is predicted herein, and supported by theoretical binding calculations that enhanced binding to Domain I of HER3 and HER1 will be achieved by extending the N-terminus of the HER ligand variant in accordance with the invention.

### Biological Activity of HER Ligand Variants and Pan-HER Antagonists

The HER ligand variants and Pan-HER antagonists of the present invention can be assayed for inhibition of HER-mediated bioactivity in one or more cell lines using a number of known methods, assays, devices and kits well known in the art.

In one embodiment of the invention the one or more cell lines comprises a cancer cell line. Cancer cell lines include, but are not limited to lung, breast, liver, heart, bone, blood, colon, brain, skin, kidney, pancreatic, ovarian, uterine and prostate or any cells isolated from tissues or tumors of the cancers listed herein.

In one embodiment of the invention are methods of identifying anticancer agents comprising assaying therapeutic Pan-HER antagonists and HER ligand variants designed by the methods described herein in a tumor xenograft system wherein a measured reduction in tumor growth rate, tumor size or tumor metastasis represents a positive hit as a candidate cancer therapeutic.

In one embodiment the disease associated with HER-mediated biological activity is a tumor. In particular the tumor is a solid tumor and/or blood or lymphatic node cancer. More specifically, tumors which can be of epithelial or mesodermal origin, can be benign or malignant types of tumors in organs such as lungs, prostate, urinary bladder, kidneys, esophagus, stomach, pancreas, brain, ovaries, skeletal system, with adenocarcinoma of breast, prostate, lungs and intestine, bone marrow cancer, melanoma, hepatoma, ear-nose-throat tumors in particular being explicitly preferred as members of so-called malignant tumors.

According to the invention, the group of blood or lymphatic node cancer types includes all forms of leukemias (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia) and lymphomas.

Examples of mesenchymal malignant tumors (so-called bone and soft-tissue sarcomas) are: fibrosarcoma; malignant histiocytoma; liposarcoma; hemangiosarcoma; chondrosarcoma and osteosarcoma; Ewing sarcoma; leio- and rhabdomyosarcoma, synovialsarcoma; carcinosarcoma.

Also contemplated within the scope of the invention are neoplasms. Neoplasms include: bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of head and throat, of the central nervous system, neoplasms of the hearing organ, pelvis, respiratory tract and urogenital tract.

In another embodiment the cancerous disease or tumor being treated or prevented is selected from the group of: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs, comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube, tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancies such as Kaposi sarcoma, AIDS-associated lymphomas, AIDS-associated lymphomas of the central nervous system, AIDS-associated Hodgkin disease, and AIDS-associated anogenital tumors, transplantation-related malignancy, metastasized tumors comprising brain metastases, lung metastases, liver metastases, bone metastases, pleural and pericardial metastases, and malignant ascites.

According to the present invention, the biological activity being assayed includes, but is not limited to; a receptor-mediated pathology such as any of the diseases or conditions noted herein, receptor-mediated cell signaling, cell growth, cell proliferation and tumor growth.

As used herein the term “receptor-mediated” refers to any phenomenon or condition, the occurrence of which can be linked or traced to the function or activity of a receptor, as that term is defined herein.

In one embodiment of the invention the inhibited biological activity is a receptor-mediated pathology selected from the group consisting of cancer (including all those identified hereinabove), inflammation, cardiovascular disease, hyperlipidemia, glucose dysregulation, epilepsy, allergies, Alzheimers disease, metabolic syndrome, cortisol resistance, Crohn disease and Huntington disease.

In one embodiment of the invention, the inhibited biological activity is receptor-mediated cell signaling. This inhibition of receptor-mediated cell signaling may result in ablation of downstream signaling by a receptor and this effect can be determined by measuring altered phosphorylation states of one or more proteins.

According to the present invention, inhibition of receptor-mediated cell signaling can be measured using autophosphorylation assays or gene expression assays. Methods of measuring and quantifying cell signaling cascades are known in the art as are methods to measure gene expression either by measuring mRNA (e.g., RT-PCR) or measuring protein levels (e.g., Western blot analysis).

It is within the scope of the present invention to design therapeutic Pan-HER antagonists that are capable of activity which is panoramic (i.e., has an effect of the same kind on multiple receptors) over two or more receptors. Further, the level or degree panoramic inhibition of biological activity may be or is substantially the same against said two or more HERs. Identification of panoramic capacity of any Pan-HER antagonist or HER ligand variant simply involves assaying the Pan-HER antagonist or HER ligand variant for inhibition of biological activity against the two or more receptors of interest.

The Pan-HER antagonists and HER ligand variants of the invention possess a number of uses. For example, the Pan-HER antagonists of the present invention can be used to treat patients wherein dysregulation of cell signaling is implicated in the pathological process of disease (e.g. cancer, inflammation). Not only may the molecules of the present invention be administered as amino-acid based molecules, they may also be administered as nucleic acid molecules in the context of gene therapy. Furthermore, these molecules may be used in diagnostic applications as well as to further basic research.

### Therapeutic Formulation and Delivery

The present invention also pertains to pharmaceutical compositions comprising the therapeutic Pan-HER antagonists described herein. For instance, a Pan-HER antagonist of the invention can be formulated with a pharmaceutically acceptable carrier or excipient to prepare a pharmaceutical composition. The carrier and composition can be sterile. The formulation should suit the mode of administration. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylase or starch, dextrose, magnesium stearate, talc, silicic acid, viscous paraffin, perfume oil, fatty acid esters, hydroxymethylcellulose, polyvinyl pyrrolidone, etc., as well as combinations thereof. In addition, carriers such as liposomes and microemulsions may be used. The Pan-HER antagonists of the invention may also be covalently attached to a protein carrier such as albumin, or a polymer, such as polyethylene glycol so as to minimize premature clearing of the polypeptides. The pharmaceutical preparations can, if desired, be mixed with auxiliary agents, e.g. lubricants, preservatives, stabilizers, wetting agents, emulsifiers, salts for influencing osmotic pressure, buffers, coloring, flavoring and/or aromatic substances and the like that do not deleteriously react with the active agent in the composition (i.e., a polypeptide and/or nucleic acid molecule of the invention).

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrrolidone, sodium saccharine, cellulose, magnesium carbonate, etc.

Methods of introduction of these compositions include, but are not limited to, transdermal, intramuscular, intraperitoneal, intraocular, intravenous, subcutaneous, pulmonary, topical, oral and intranasal. In one embodiment, topical applications include those for treating conditions such as scarring, skin cancer and psoriasis.

Other suitable methods of introduction can also include gene therapy (as described below), rechargeable or biodegradable devices, particle acceleration devices (“gene guns”) and slow release polymeric devices. The pharmaceutical compositions of this invention can also be administered as part of a combination therapy with other Pan-HER antagonists or other compounds.

The Pan-HER antagonists of the present invention can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentration in a hermetically sealed container such as an ampoule or sachette indicating the quantity of active compound (polypeptide and/or nucleic acid). Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

The Pan-HER antagonists described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The Pan-HER antagonists of the invention are administered in a therapeutically effective amount. The amount of Pan-HER antagonist that will be therapeutically effective in the treatment of a particular disorder or conditions will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of the disease or condition, and should be decided according to the judgment of a practitioner and each patient's circumstances. Effective doses may be extrapolated from dose-response curves derived from in vitro or animal model test systems.

The present invention also pertains to methods of treatment (prophylactic, diagnostic, and/or therapeutic) for conditions characterized by HER-mediated pathology, HER overexpression, or dysregulation of cell signaling. A “condition characterized by dysregulation of cell signaling” is a condition in which the presence of a Pan-HER antagonist of the invention is therapeutic. Such conditions include many types of cancer. Dysregulation of cell signaling has also been implicated in a variety of other disorders. The present invention also features a method of treating a condition characterized by HER over-expression or HER ligand-mediated pathology in a patient, comprising administering to the patient a therapeutically effective amount of a pharmaceutical composition comprising at least one Pan-HER antagonist of the invention. A single Pan-HER antagonist specific for HER1, HER3 and HER4 is very desirable as it provides a powerful therapeutic for targeting and treating diseases (such as cancer) in which undesirable HER overexpression, overexpression of HER ligands or other HER-mediated biological activity of one or more HER family members is implicated.

The term “treatment” as used herein, refers not only to ameliorating symptoms associated with the disease or condition, but also preventing or delaying the onset of the disease, and also lessening the severity or frequency of symptoms of the disease or condition. More than one Pan-HER antagonist of the present invention can be used concurrently as a co-therapeutic treatment regimen, if desired. As used herein, a “co-therapeutic treatment regimen” means a treatment regimen wherein two drugs are administered simultaneously, in either separate or combined formulations, or sequentially at different times separated by minutes, hours or days, but in some way act together to provide the desired therapeutic response. The Pan-HER antagonists of the invention may also be used in conjunction with other drugs that inhibit various aberrant activities of HER-mediated pathologies or dysregulated cell signaling. Such additional drugs include but are not limited to receptor specific antibodies, small molecule receptor inhibitors, and traditional chemotherapeutic agents.

The therapeutic compound(s) of the present invention are administered in a therapeutically effective amount (i.e., an amount that is sufficient to treat the disease or condition, such as by ameliorating symptoms associated with the disease or condition, preventing or delaying the onset of the disease or condition, and/or also lessening the severity or frequency of symptoms of the disease or condition). The amount that will be therapeutically effective in the treatment of a particular individual's disease or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or condition, and should be decided according to the judgment of a practitioner and each patient's circumstances. Effective doses may be extrapolated from dose-response curves derived from in vitro or animal model test systems.

A therapeutically effective amount of a Pan-HER antagonist of this invention is typically an amount of Pan-HER antagonist such that when administered in a physiologically tolerable composition is sufficient to achieve a plasma concentration of from about 0.1 microgram (ug) per milliliter (ml) to about 100 ug/ml, preferably from about 1 ug/ml to about 5 ug/ml, and usually about 5 ug/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

Dosages may also be based on the range of serum levels of EGF (0.1-1 ng/ml) and/or relative to the affinity for the ACL. Using this starting point, compounds of the invention may be administered in doses up to ten-fold these measurements. For example, if the ACL affinity is 10 nM and the affinity of EGF is 1 nM, then the dosing range would be between about 10 ng/mL and about 100 ng/mL.

The therapeutic compositions containing a Pan-HER antagonist or a polypeptide of this invention may be administered via a unit dose. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The therapeutic compounds of the present invention can be used either alone or in a pharmaceutical composition as described above. For example, the gene for a Pan-HER antagonist or HER ligand variant of the present invention, either by itself or included within a vector, can be introduced into cells (either in vitro or in vivo) such that the cells produce the desired Pan-HER antagonist polypeptide. If desired, cells that have been transfected with the nucleic acid molecule of the present invention can be introduced (or re-introduced) into an individual affected with the disease.

### Gene Therapy

The therapeutic Pan-HER antagonist of the present invention may also be used in the context of gene therapy. In the meaning of the invention, “gene therapy” is a form of treatment using natural or recombinantly engineered nucleic acid constructs, single gene sequences or complete gene or chromosome sections or encoded transcript regions, derivatives/modifications thereof, with the objective of a biologically based and selective inhibition or reversion of disease symptoms and/or the causal origin thereof.

For example, gene therapy may be effected using suitable vectors such as viral vectors or/and complex formation with lipids or dendrimers. Gene therapy may also proceed via packaging in protein coats. Furthermore, the polynucleotide can be fused or complexed with another molecule supporting the directed transport to the target site, uptake in and/or distribution inside a target cell. The kind of dosage and route of administration can be determined by the attending physician according to clinical requirements. As is familiar to those skilled in the art, the kind of dosage will depend on various factors, such as size, body surface, age, sex, or general health condition of the patient, but also on the particular agent being administered, the time period and type of administration and on other medications possibly administered in parallel, especially in a combination therapy.

The therapeutic Pan-HER antagonists of the invention may also be contained within a kit. As such, the invention also relates to a kit comprising the therapeutic Pan-HER antagonist and/or the pharmaceutical composition. Furthermore, the invention also relates to an array comprising the therapeutic Pan-HER antagonist and/or the pharmaceutical composition. Kits and arrays can be used in the diagnosis and/or therapy of diseases associated with the dysregulation of cell signaling. The invention also relates to the use of said therapeutic Pan-HER antagonist, said kit, said array in the diagnosis, prophylaxis, reduction, therapy, follow-up and/or aftercare of diseases associated with an HER-mediated pathology or dysregulation of cell signaling.

### EXAMPLES

### Example 1

### Methods and Reagents

Cloning and gene expression. The human epidermal growth factor gene (EGF) was synthesized chemically and ligated into the Pet-9a vector (Novagen) at the NdeI and BamHI cloning sites. The EGF gene contained the OmpA leader sequence followed by an N-terminal 6×-his tag (underlined) and a factor Xa cleavage site for future his-tag removal, (BOLDED: IEGR) if necessary, and corresponds to the following amino acid sequence:

| | SEQ |
|---|---|
| | ID |
| EGF gene clone | NO |
| MKKTAIAIAVALAGFATVAQAHHHHHHIEGRNSDSECPLSHDGYCLH | 1 |
| DGVCMYIEALDKYACNCWGYIGERCQYRDLKWWELR | |

This original clone, designated pMLPP1, was used as a basis for cloning all Pan HER ligand variants (including substitution, deletion, insertion and domain swap variants) using the QuickChange mutagenesis kit (Stratagene). For protein production the EGF plasmids were transformed into E. coli strain BL21 (DE3) pLysS (Novagen).

Production of ligand variants. Single colonies were inoculated into shake flask cultures containing 15 ml LB+Km25+Cm30. After growth overnight, samples of culture were frozen for stocks, and for plasmid preps to confirm the identities of the EGF variant gene inserts. The remaining cultures were used to inoculate production cultures in Terrific Broth+Km25+Cm30. Cells were induced with 0.2 mM IPTG during early log phase, and the cultures were grown overnight. Culture supernatants were collected by centrifugation and production was confirmed by dot blot using the Mouse Western Breeze Chromogenic Immunodection System (Invitrogen cat#WB7103) with primary antibody: 1:1000 mouse anti-penta his antibody (Qiagen cat#34660).

EGF protein purification. Three ml of Ni-NTA resin (Qiagen #30230) was used to pack 5 ml columns (Qiagen cat#34964) which were equilibrated with PBS pH 8.0. Culture supernatants were adjusted to pH 7.5-8.0 with 1N HCL before loading on columns. Columns were washed with PBS and PBS+10 mM imidazole; EGF variant proteins were eluted from columns with PBS+250 mM imidazole. Bradford protein assays were used to monitor protein concentrations.

Protein concentrate and buffer exchange. Column eluents were dialyzed in PBS at 4° C. with one buffer exchange, and then concentrated with 3000 MWCO Macrosep centrifuge devices (ISC# OD003C41). The final product was tested for protein concentration using the BCA method and for purity by SDS-PAGE.

### Example 2

### Design and Validation of Pan-HER Antagonists

### Selection of Druggable Ligand and Domain Binding Optimization

Using native EGF as a starting druggable ligand, three N-terminal modification variants were created which improve binding. These modifications alter binding to HER3 with no effect on EGFR (HER1). The variants are listed in Table 3.

TABLE 3
EGF ligand variant

| EGF ligand | | |
|---|---|---|
| variant | N-terminal modification | SEQ ID NO |
| BiRegulin | Amino terminal residues | 2 |
| (BiR) | (NSDSE) are replaced with the | |
| | corresponding residues of | |
| | heregulin (SHLVK) | |
| WVS | Amino acids 2 and 3 are | 3 |
| | replaced with W and V | |
| | respectively, resulting in a | |
| | modified N-terminus sequence | |
| | of (NWVSE) | |
| T1E | Amino terminal residues | 4 |
| | (NSDSE) are replaced with | |
| | seven residues from TGF-α | |
| | (VVSHFND). N-terminal | |
| | domain swap with TGF-alpha. | |

To the modified druggable ligands of Table 3, further modifications were then made, which abrogate binding to Domain III in both EGFR and HER3. These modified ligands are listed in Table 4.

TABLE 4
Ligands modified to inhibit domain binding

| | | | SEQ ID |
|---|---|---|---|
| Ligand Variant | Background | Description | NO |
| wvs-R41DL47G | WVS | Amino acid R at position | 5 |
| | | 41 replaced by D; amino | |
| | | acid L at position 47 | |
| | | replaced by G | |
| wvs-R41D | WVS | Amino acid R at position | 6 |
| | | 41 replaced by D | |
| wvs-L47G | WVS | Amino acid L at position | 7 |
| | | 47 replaced by G | |

### Example 3

### Use of Phage Display Vectors to Produce and Assay Pan-HER Antagonists

### Library Construction and Phage Panning

Two libraries were constructed using the Kunkel procedure. Random clones were sequenced from each library and it was calculated that each nucleic acid variant was represented between 500 and 1000 times and each amino acid sequence variant was represented between 10⁴-10⁵ times.

As a starting point, the libraries were constructed to contain the modified agonists and antagonists or combinations thereof from Example 2 in addition to alterations in the B-loop of EGF, which is known to be critical for binding to Domain I of the EGF receptor, at either residues 21-25 or 26-30. A selection of members from the libraries are shown in Tables 5-8.

TABLE 5
Library PD1B: Residues 21-35: first half of
B-loop Preamplification

| | | | SEQ |
|---|---|---|---|
| Ligand | | Amino acid | ID |
| Variant | Codon Sequence | sequence | NO |
| wvs-R41DL47G | ATG TAT ATT GAA GCG | MYIEA | 5 |
| PD1B-25 | CGT GCG CTA GCG AGG | RAVAR | 8 |
| PD1B-26 | AAG AAT TAT AAT GAG | KNYNE | 9 |
| PD1B-29 | TAT ATG AAG GGG GGG | YAKGG | 10 |
| PD1B-34 | GGT GGG GGG AAG GCG | GGGKA | 11 |
| PD1B-37 | GGT GGG TCG AAG GGG | GGSKG | 12 |
| PD1B-40 | AGG GAG AGG ACG GGT | RERTG | 13 |
| PD1B-33 | CCG CGG ACT GCT CCG | PRTAP | 14 |

TABLE 6
Library PD1B: Residues 21-35: first half of
B-loop Amplified

| | | | SEQ |
|---|---|---|---|
| Ligand | | Amino acid | ID |
| Variant | Codon Sequence | sequence | NO |
| wvs-R41DL47G | ATG TAT ATT GAA GCG | MYIEA | 5 |
| PD1B-41 | ACG ACG CAG ACG CCG | TTQTP | 15 |
| PD1B-42 | ACG AAT AAG GAG AGG | TNKER | 16 |
| PD1B-43 | TCG GGG AGG CCG ACG | SGRPT | 17 |
| PD1B-44 | ATG GGT ATG GGG CGG | MGMGR | 18 |
| PD1B-45 | ATG GGG AGT TGC GGG | MGSSG | 19 |
| PD1B-46 | ACG ACG AAT AAG GCG | TTNKA | 20 |
| PD1B-47 | AAG CCG GAG AAG CAG | KPEKQ | 21 |
| PD1B-50 | GAT AAT CCG ATG CGT | DNPMR | 22 |
| PD1B-52 | GGG CCG CAG GCT CCT | GPQAP | 23 |

TABLE 7
Library PD2B: Residues 26-30: second half of
B-loop Preamplification

| | | | SEQ |
|---|---|---|---|
| Ligand | | Amino acid | ID |
| Variant | Codon Sequence | sequence | NO |
| wvs-R41DL47G | CTG GAT AAA TAT GCG | LDKYA | 5 |
| PD2B-37 | CAT CCC AAG TCT TAT | HPKSY | 24 |
| PD2B-38 | ACT CCT TCT TAT TTG | TPSYL | 25 |
| PD2B-39 | AAT CGC GAG AAG ACT | NREKT | 26 |
| PD2B-40 | AGT AAG CGT CAG CCG | SKRQP | 27 |
| PD2B-41 | CAG ATT AAG CTT CTG | QIKLL | 28 |
| PD2B-44 | GGG ACT AAG CAT CGG | GTKHR | 29 |
| PD2B-45 | ATT AGC TTG CGG CCT | ISLRS | 30 |
| PD2B-47 | GGG ACT GCG CGT CCT | GTARG | 31 |
| PD2B-48 | GAG AAT AAG CGT CCT | ENKRR | 32 |

TABLE 8
Library PD2B: Residues 26-30: second half of
B-loop Amplified

| | | | SEQ |
|---|---|---|---|
| Ligand | | Amino acid | ID |
| Variant | Codon Sequence | sequence | NO |
| wvs-R41DL47G | CTG GAT AAA TAT GCT | LDKYA | 5 |
| PD2B-49 | TAT GGT AAT ACT ACG | YGNTT | 33 |
| PD2B-50 | GAT CGG TCT CTT ACG | DRSLT | 34 |
| PD2B-51 | TCG CAT GGG CAG GAG | SHGQE | 35 |
| PD2B-52 | CAT ATT GCT GGT GCT | QIAGA | 36 |
| PD2B-53 | CCT AAT CCT AGT CCG | PNPSP | 37 |
| PD2B-54 | GGT AAG TCG AGT AAG | GKSMK | 38 |
| PD2B-55 | CAG CCG CAT TTG TCT | QPHLS | 39 |
| PD2B-56 | CCT CAC GCG TCT CTT | PHASL | 40 |
| PD1B-59 | CAG ATG CAG TCG CGT | QMQSR | 41 |

Phage panning was performed according to the teachings of Rodi and Malowski, (Curr. Opin. Biotechnol., 10:87-93; 1999). Briefly, genes encoding modified ligands were cloned into the pentavalent M13 phage display system (New England Biolabs). Sequences included those coding for the three pan-HER agonists (T1E, WVS, and BiR); those coding for ligands having the agonist modifications in addition to modifications that reduce binding to HER receptor domains (e.g. R41D and L47G); and those variants generated from one of two libraries constructed using the Kpn I and Eag I restriction sites of the M13KE phage vector for expression as an N-terminus-fusion with the pIII coat protein of the M13 phage.

All five copies of pIII should display the cloned protein. To produce phage, the vector with insert was transformed into electrocompetent E. coli 10GF′. Transformation outgrowth was used to infect E. coli and infected cells were plated on LB+tet20+xgal+IPTG. Blue plaques resulting from the infection were amplified and plasmid DNA was sequenced to verify the identity of the insert. Phage were amplified by infecting E. coli in LB culture, and cells were removed by centrifugation. Phage were harvested by PEG precipitation. These phage were used to measure binding affinity of the ligand variants as well as biological activity by stimulation of HER receptor dependent cell proliferation.

### Phage ELISA for Analysis of Binding Affinity

A431 cells for EGFR binding or T47D cells for HER3 binding were grown as monolayers in tissue culture flasks in media containing fetal bovine serum. Cells were trypsinized, neutralized with growth medium, washed twice with DPBS and resuspended in ice-cold PBS-Glu-T. 10₅ cells were transferred to 96 well plates and incubated on ice for 1 hour in the presence of varied concentrations of phage. Cells were centrifuged and washed 5× with PBS-T then incubated for one hour at room temperature with anti-M13 pVIII coat protein antibody conjugated with horseradish peroxidase (HRP). Cells again centrifuged and washed 5× with PGS-T. Color developed with TMP followed by H₂SO₄. Cells pelleted and supernatant transferred to optically transparent plate for measurement of absorbance at 450 nm.

Phage particles displaying ligand variants were evaluated for binding affinity to the HER1 receptor (EGFR) in T47D whole cell suspension by measuring absorbance at Abs450. Theoretical estimates were also performed. The results are shown in Table 9. “N.D.” indicates not determined. EC50 is the concentration of phage necessary for a 50% stimulation of cell proliferation. From the binding curves it is evident that ligand binding was greatly attenuated by the wvs-R41DL47G ligand variant.

TABLE 9
Binding affinity of ligand variants: T47D cells

| | Estimated binding | Calculated binding |
|---|---|---|
| Ligand | (EC50) | (EC50) |
| Variant | phage titer/mL | phage titer/mL |
| WVS | 9 × 10⁸ | 9.8 × 10⁹ |
| T1E | 1 × 10¹⁰ | ND |
| wvs-R41D | >1 × 10¹² | ND |
| wvs- | ND | 2.7 × 10²⁰ |
| R41DL47G | | |

Additional phage particles displaying ligand variants were evaluated for binding affinity to the HER3 receptor in A431 whole cell suspensions by measuring absorbance at Abs450. The results are shown in Table 10. From the binding curves it is evident that ligand binding was greatly attenuated by the wvs-R41DL47G and wvs-R41D ligand variants.

TABLE 10
Binding affinity of ligand variants: A431 cells

| | Calculated binding (EC50) |
|---|---|
| Ligand Variant | phage titer/mL |
| WVS | 6.6 × 10⁸ |
| wvs-R41D | 5.8 × 10¹¹ |
| wvs-L47G | 6.6 × 10¹⁰ |
| wvs-R41DL47G | 4.8 × 10¹² |

### Cell Lines

### A431 Cells

The human epidermoid carcinoma line, A431, were obtained from ATCC. Stock cultures of A-431 were propagated in DMEM medium containing 10% fetal bovine serum. A431 cells were used to evaluate EGFR receptor binding.

### T47D Cells

Human ductal carcinoma cells were obtained from ATCC. They were maintained in RPMI 1640 medium with 2 mM L-glutamine adjusted to contain 1.5 g/L sodium bicarbonate, 4.5 g/L glucose, 10 mM HEPES, and 1.0 mM sodium pyruvate and supplemented with 0.2 Units/ml bovine insulin, 90%; fetal bovine serum, 10%. T47-D cells were used to evaluate HER3 receptor binding.

### Example 4

### Biological Activity of Phage-Fusions

It was unexpectedly discovered herein that, not only could phage particles be used to measure binding affinity, but that these same particles displaying the ligand variants of the invention could also be used directly in assays to determine biologic activity.

Phage-fusion particles displaying ligand variants were evaluated for their ability to stimulate cell proliferation in the cell proliferation assay described herein in both an EGF dependent cell line, HER5 and a heregulin dependent cell line, MCF-7. The data are summarized in Tables 11 and 12.

TABLE 11
Cell Proliferation: HER5 cells

| | Calculated cell proliferation |
|---|---|
| Ligand Variant | (EC50; picomolar) |
| EGF (purified protein) | 1150 |
| BiR (phage) | 3.2 |
| T1E (phage) | 2.6 |
| WVS (phage) | 4.9 |
| T1ER41D (phage) | 2800 |

TABLE 12
Cell Proliferation: MCF-7

| | Calculated cell proliferation |
|---|---|
| Ligand Variant | (EC50; picomolar) |
| Heregulin (purified protein) | 6151 |
| T1E (phage) | 3052 |
| WVS (phage) | 237 |
| T1ER41D (phage) | >10⁶ |

It is known that HER5 cells can be stimulated by EGF and BiR but not by HRG, while MCF-7 cells can be stimulated by BiR and HRG but not by EGF. It has also previously been demonstrated using isolated ligand variants that the pan-HER agonists T1E, WVS, and BiR, are all capable of stimulating cell proliferation in EGFR-dependent HER5 cells while the weak binding mutant T1ER41D has greatly attenuated activity and that MCF-7's are stimulated most effectively by WVS and very weakly by BiR.

Here it is demonstrated that it is possible to use phage-fusions directly in determining these same parameters. It should be noted that WVS and T1E phage are more potent than the purified protein ligands of EGFR and HER3. This is due to the pentavalent state of the phage fusions which results in increased apparent affinity due to avidity effects.

### Cell Lines and Cell Proliferation Assay

### HER5 Cells

The HER5 cell line, a murine fibroblast line (derived from the NR-6 line; mouse fibroblast cells that overexpress human EGFR) that has been stably transfected to express the human EGF receptor was provided by Dr. M. C. Hung (MD Anderson Cancer Center).

Stock cultures of HER5 were propagated in D-MEM/F12 medium containing 10% fetal bovine serum, 100 units/ml of penicillin and 100 ug/ml of streptomycin in a water-jacketed incubator at 37° C. in a humidified 5% CO₂ atmosphere.

For HER5 proliferation assays, the cells were changed into DMEM/F12 without serum for 24 hours. Cells were then trypsinized and suspended at 1E5 cells/ml. Serial dilutions of EGF (PeproTech, Rocky Hill, N.J.), and HER ligand polypeptide variants were prepared in serum-free DMEM/F12 at 2-fold the final concentration and plated into the wells of 96-well plates. Fifty microliters of cell suspension (5000 cells) were added to appropriate wells bringing the total volume to 100 ul at the desired concentrations. Plates were incubated for a 48 hour proliferation period. Cell proliferation was determined by addition of 10 ul/well of WST-1 Cell Proliferation Reagent (Roche Applied Sciences, Indianapolis, Ind.) for the last three hours of the proliferation period. WST-1 is a tetrazolium salt that is cleaved to formazan dye by mitochondrial dehydrogenases in viable cells. The amount of formazan was measured at 450 nm using a microplate reader (Dynex Technologies) with MRX Revelation software.

### MCF-7 Cells

MCF-7 cells (human breast cancer cell lines that express HER2 and HER3) were obtained from the American Type Culture Collection (ATCC). Stock cultures of MCF-7 were maintained in Eagle's MEM supplemented with 1% ITS-X (Invitrogen) and 10% fetal bovine serum.

For proliferation assays, MCF-7 cells were transferred to serum-free medium (SFM) for 24 hours and then trypsinized and suspended at 10⁵ cells/mL in SFM. Fifty microliters of cell suspension (5000 cells) were plated per well in 96 well microtiter plates. Serial dilutions of HER ligands or mutant proteins were prepared at twice the final concentration in SFM and 50 ul was added to wells, bringing the final volume to 100 ul at the desired final concentration. Plates were incubated for 72 hours at 37 C in a humidified 5% CO2 atmosphere. Cell proliferation was determined by addition of 10 ul/well of WST-1 Cell Proliferation Reagent (Roche Applied Sciences, Indianapolis, Ind.) for the last three hours of the proliferation period.

### Example 5

### Domain Swap to Alter Selectivity

A domain swap was undertaken within the B-loop of EGF (residues 21-30). This swap was expected to further enhance ligand variant binding, particularly to Domain I of the EGF receptor and HER3 receptor. The first half of the B-loop, (amino acid residues 21-25), and the second half of the B-loop (residues 26-30) were rationally redesigned to produce the variants in Table 13. The variants, D4, D4-2 and E8 were all prepared on the WVS background.

The phage fusion ligand variants were then evaluated for binding using the assay described herein in both A431 cells (to investigate EGFR binding) and T47D cells (to investigate HER3 receptor binding) and EC50s were calculated. Binding data are shown in Tables 14 and 15.

TABLE 13
Ligand variants

| Ligand | | First half/ | SEQ |
|---|---|---|---|
| Variant | B-loop Sequence | second half | ID |
| WVS | MYIEALDKYA | Wild type/ | 3 |
| | | Wild type | |
| WVS- | MYIEALDKYA | Wild type/ | 5 |
| R41DL47G | | Wild type | |
| D4 | MYIEAYRVKT | Wild type/ | 42 |
| | | YRVKT | |
| D4-2 | YRVKTLDKYA | YRVKT/ | 43 |
| | | Wild type | |
| E8 | MYIEATKYRG | Wild type/ | 44 |
| | | TKYRG | |

TABLE 14
Binding of ligand variants (First half loop)

| | Calculated binding | Calculated binding |
|---|---|---|
| | (EC50) in A431 cells | (EC50) in T47D cells |
| Ligand Variant | phage titer/mL | phage titer/mL |
| WVS | 1.9 × 10⁹ | 7.5 × 10⁹ |
| WVS-R41DL47G | 2.8 × 10¹⁰ | 1.1 × 10¹² |
| D4-2 | 3.6 × 10¹⁰ | 8.6 × 10¹⁰ |

The D4-2 ligand variant having a half-loop modification (YRVKT) in the first half was determined to bind only the HER3 receptor and is therefore not panoramic to multiple EGF receptors. Consequently, D4-2 is a HER3 specific antagonist.

TABLE 15
Binding of ligand variants (Second half loop)

| | Calculated binding | Calculated binding |
|---|---|---|
| | (EC50) in A431 cells | (EC50) in T47D cells |
| Ligand Variant | phage titer/mL | phage titer/mL |
| WVS | 1.4 × 10⁹ | 7.6 × 10⁹ |
| WVS-R41DL47G | 4.0 × 10¹⁰ | 2.6 × 10¹¹ |
| D4 | 3.9 × 10⁹ | 2.9 × 10¹⁰ |
| E8 | 5.2 × 10⁹ | 3.1 × 10¹⁰ |

Binding curves and EC50 calculations show that the D4 and E8 variants have intermediate binding properties for both receptors between that of the WVS variant and the WVS-R41DL47G variant.

Together these data indicate that the half-loop modification (swap) can yield ligands with improved binding properties (compare WVS-R41DL47G having a wild type B-loop with D4 having a modified second half loop). Furthermore, it is demonstrated that by moving the half loop modification found to improve Domain I binding in D4 from residues 26-30 to residues 21-25 in the B-loop producing variant D4-2, binding can be selectively enhanced for one receptor over another. It is also contemplated that using this method, receptor binding may be a titratable property in the optimization of therapeutic ligands.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A human epidermal receptor (HER) ligand variant, said variant having a T1E or WVS background and wherein at least one amino acid corresponding to G18, G39, R41 or L47 of human wild-type epidermal growth factor (EGF) is substituted with a different amino acid.

2. The HER ligand variant of claim 1, which is a Pan-HER antagonist.

3. The HER ligand variant of claim 1, wherein the amino acid G18 is substituted with glutamate (G18E), glutamine (G18Q), lysine (G18K), phenylalanine (G18F), or leucine (G18L).

4. The HER ligand variant of claim 1 further comprising an amino acid substitution at the position corresponding to V35 of wild-type EGF wherein the amino acid V35 is substituted with glutamate (V35E).

5. The HER ligand variant of claim 1, wherein the amino acid G39 is substituted with glutamate (G39E), glutamine (G39Q), lysine (G39K), aspartic acid (G39D) or isoleucine (G391), leucine (G39L) or phenylalanine (G39F).

6. The HER ligand variant of claim 1, wherein the amino acid R41 is substituted with aspartate (R41D).

7. The HER ligand variant of claim 1, wherein the amino acid L47 is substituted with glycine (L47G), apartate (L47D) or arginine (L47R).

8. The HER ligand variant of claim 1 selected from T1E-G39L, T1E-R41D, T1E-L47G, T1E-R41DL47G, WVS-G39L, WVS-R41D and WVS-L47G, and WVS-R41 DL47G.

9. The HER ligand variant of claim 2, wherein the Pan-HER antagonistic activity is panoramic against at least two members selected from the group consisting of HER1, HER3 and HER4.

10. The HER ligand variant of claim 1 having a WVS background and wherein the amino acid position that corresponds to amino acid L47 of human wild-type epidermal growth factor (EGF) is substituted with another amino acid and wherein the amino acid position that corresponds to amino acid R41 of human wild-type epidermal growth factor (EGF) is substituted with another amino acid.

11. The HER ligand variant of claim 10 having at least one of a substituted feature, modified feature, or a substituted and modified feature.

12. The HER ligand variant of claim 11, wherein the HER ligand variant is a Pan-HER antagonist.

13. A pharmaceutical composition comprising the HER ligand variant of claim 8 and a pharmaceutically acceptable carrier.

14. A method of treating a patient with a disease characterized by overexpression of HER comprising, administering to the patient, a therapeutically effective amount of a pharmaceutical composition of claim 13.

15. The method of claim 14, wherein the disease is cancer or psoriasis.

16. The method of claim 15, wherein the cancer is selected from the group consisting of gliomas, squamous cell carcinomas, breast carcinomas, melanomas, invasive bladder carcinomas, colorectal carcinomas and esophageal cancers.


SEQUENCE LISTING (97 sequences)

SEQ ID NO: 1 - protein, 83 aa, Artificial Sequence
  EGF Clone
MKKTAIAIAVALAGFATVAQAHHHHHHIEGRNSDSECPLSHDGYCLHDGVCMYIEALDKYACNCWGYIGERCQYRDLKWWELR

SEQ ID NO: 2 - protein, 53 aa, Artificial Sequence
  Ligand Variant
SHLVKCPLSHDGYCLHDGVCMYIEALDKYACNCVVGYIGERCQYRDLKWWELR

SEQ ID NO: 3 - protein, 53 aa, Artificial Sequence
  Ligand Variant
NWVSECPLSHDGYCLHDGVCMYIEALDKYACNCVVGYIGERCQYRDLKWWELR

SEQ ID NO: 4 - protein, 55 aa, Artificial Sequence
  Ligand Variant
VVSHFNDCPLSHDGYCLHDGVCMYIEALDKYACNCVVGYIGERCQYRDLKWWELR

SEQ ID NO: 5 - protein, 53 aa, Artificial Sequence
  Ligand Variant
NWVSECPLSHDGYCLHDGVCMYIEALDKYACNCVVGYIGEDCQYRDGKWWELR

SEQ ID NO: 6 - protein, 53 aa, Artificial Sequence
  Ligand Variant
NWVSECPLSHDGYCLHDGVCMYIEALDKYACNCVVGYIGEDCQYRDLKWWELR

SEQ ID NO: 7 - protein, 53 aa, Artificial Sequence
  Ligand Variant
NWVSECPLSHDGYCLHDGVCMYIEALDKYACNCVVGYIGERCQYRDGKWWELR

SEQ ID NO: 8 - protein, 5 aa, Artificial Sequence
  Ligand Variant
RAVAR

SEQ ID NO: 9 - protein, 5 aa, Artificial Sequence
  Ligand Variant
KNYNE

SEQ ID NO: 10 - protein, 5 aa, Artificial Sequence
  Ligand Variant
YAKGG

SEQ ID NO: 11 - protein, 5 aa, Artificial Sequence
  Ligand Variant
GGGKA

SEQ ID NO: 12 - protein, 5 aa, Artificial Sequence
  Ligand Variant
GGSKG

SEQ ID NO: 13 - protein, 5 aa, Artificial Sequence
  Ligand Variant
RERTG

SEQ ID NO: 14 - protein, 5 aa, Artificial Sequence
  Ligand Variant
PRTAP

SEQ ID NO: 15 - protein, 5 aa, Artificial Sequence
  Ligand Variant
TTQTP

SEQ ID NO: 16 - protein, 5 aa, Artificial Sequence
  Ligand Variant
TNKER

SEQ ID NO: 17 - protein, 5 aa, Artificial Sequence
  Ligand Variant
SGRPT

SEQ ID NO: 18 - protein, 5 aa, Artificial Sequence
  Ligand Variant
MGMGR

SEQ ID NO: 19 - protein, 5 aa, Artificial Sequence
  Ligand Variant
MGSSG

SEQ ID NO: 20 - protein, 5 aa, Artificial Sequence
  Ligand Variant
TTNKA

SEQ ID NO: 21 - protein, 5 aa, Artificial Sequence
  Ligand Variant
KPEKQ

SEQ ID NO: 22 - protein, 5 aa, Artificial Sequence
  Ligand Variant
DNPMR

SEQ ID NO: 23 - protein, 5 aa, Artificial Sequence
  Ligand Variant
GPQAP

SEQ ID NO: 24 - protein, 5 aa, Artificial Sequence
  Ligand Variant
HPKSY

SEQ ID NO: 25 - protein, 5 aa, Artificial Sequence
  Ligand Variant
TPSYL

SEQ ID NO: 26 - protein, 5 aa, Artificial Sequence
  Ligand Variant
NREKT

SEQ ID NO: 27 - protein, 5 aa, Artificial Sequence
  Ligand Variant
SKRQP

SEQ ID NO: 28 - protein, 5 aa, Artificial Sequence
  Ligand Variant
QIKLL

SEQ ID NO: 29 - protein, 5 aa, Artificial Sequence
  Ligand Variant
GTKHR

SEQ ID NO: 30 - protein, 5 aa, Artificial Sequence
  Ligand Variant
ISLRS

SEQ ID NO: 31 - protein, 5 aa, Artificial Sequence
  Ligand Variant
GTARG

SEQ ID NO: 32 - protein, 5 aa, Artificial Sequence
  Ligand Variant
ENKRR

SEQ ID NO: 33 - protein, 5 aa, Artificial Sequence
  Ligand Variant
YGNTT

SEQ ID NO: 34 - protein, 5 aa, Artificial Sequence
  Ligand Variant
DRSLT

SEQ ID NO: 35 - protein, 5 aa, Artificial Sequence
  Ligand Variant
SHGQE

SEQ ID NO: 36 - protein, 5 aa, Artificial Sequence
  Ligand Variant
QIAGA

SEQ ID NO: 37 - protein, 5 aa, Artificial Sequence
  Ligand Variant
PNPSP

SEQ ID NO: 38 - protein, 5 aa, Artificial Sequence
  Ligand Variant
GKSMK

SEQ ID NO: 39 - protein, 5 aa, Artificial Sequence
  Ligand Variant
QPHLS

SEQ ID NO: 40 - protein, 5 aa, Artificial Sequence
  Ligand Variant
PHASL

SEQ ID NO: 41 - protein, 5 aa, Artificial Sequence
  Ligand Variant
QMQSR

SEQ ID NO: 42 - protein, 10 aa, Artificial Sequence
  Ligand Variant
MYIEAYRVKT

SEQ ID NO: 43 - protein, 10 aa, Artificial Sequence
  Ligand Variant
YRVKTLDKYA

SEQ ID NO: 44 - protein, 10 aa, Artificial Sequence
  Ligand Variant
MYIEATKYRG

SEQ ID NO: 45 - protein, 53 aa, Homo Sapien
NSDSECPLSHDGYCLHDGVCMYIEALDKYACNCVVGYIGERCQYRDLKWWELR

SEQ ID NO: 46 - protein, 53 aa, Pig
NSYSECPPSHDGYCLHGGVCMYIEAVDSYACNCVFGYVGERCQHRDLKWWELR

SEQ ID NO: 47 - protein, 52 aa, Cat
NSYQECPPSYDGYCLYNGVCMYIEAVDRYACNCVFGYVGERCQHRDLKWELR

SEQ ID NO: 48 - protein, 52 aa, Dog
NGYRECPSSYDGYCLYNGVCMYIEAVDRYACNCVFGYVGERCQHRDLKWELR

SEQ ID NO: 49 - protein, 53 aa, Mouse
NSYPGCPSSYDGYCLNGGVCMHIESLDSYTCNCVIGYSGDRCQTRDLRWWELR

SEQ ID NO: 50 - protein, 48 aa, Horse
NSYQECSQSYDGYCLHGGKCVYLVQVDTHACNCVVGYVGERCQHQDLR

SEQ ID NO: 51 - protein, 53 aa, Rat
NSNTGCPPSYDGYCLNGGVCMYVESVDRYVCNCVIGYIGERCQHRDLRWWKLR

SEQ ID NO: 52 - protein, 15 aa, Artificial Sequence
  Ligand Variant
ATGTATATTGAAGCG

SEQ ID NO: 53 - protein, 5 aa, Artificial Sequence
  Ligand Variant
MYIEA

SEQ ID NO: 54 - protein, 15 aa, Artificial Sequence
  Ligand Variant
CGTGCGCTAGCGAGG

SEQ ID NO: 55 - protein, 15 aa, Artificial Sequence
  Ligand Variant
AAGAATTATAATGAG

SEQ ID NO: 56 - protein, 15 aa, Artificial Sequence
  Ligand Variant
TATATGAAGGGGGGG

SEQ ID NO: 57 - protein, 15 aa, Artificial Sequence
  Ligand Variant
GGTGGGGGGAAGGCG

SEQ ID NO: 58 - protein, 15 aa, Artificial Sequence
  Ligand Variant
GGTGGGTCGAAGGGG

SEQ ID NO: 59 - protein, 15 aa, Artificial Sequence
  Ligand Variant
AGGGAGAGGACGGGT

SEQ ID NO: 60 - protein, 15 aa, Artificial Sequence
  Ligand Variant
CCGCGGACTGCTCCG

SEQ ID NO: 61 - protein, 15 aa, Artificial Sequence
  Ligand Variant
ACGACGCAGACGCCG

SEQ ID NO: 62 - protein, 15 aa, Artificial Sequence
  Ligand Variant
ACGAATAAGGAGAGG

SEQ ID NO: 63 - protein, 15 aa, Artificial Sequence
  Ligand Variant
TCGGGGAGGCCGACG

SEQ ID NO: 64 - protein, 15 aa, Artificial Sequence
  Ligand Variant
ATGGGTATGGGGCGG

SEQ ID NO: 65 - protein, 15 aa, Artificial Sequence
  Ligand Variant
ATGGGGAGTTGCGGG

SEQ ID NO: 66 - protein, 15 aa, Artificial Sequence
  Ligand Variant
ACGACGAATAAGGCG

SEQ ID NO: 67 - protein, 15 aa, Artificial Sequence
  Ligand Variant
AAGCCGGAGAAGCAG

SEQ ID NO: 68 - protein, 15 aa, Artificial Sequence
  Ligand Variant
GATAATCCGATGCGT

SEQ ID NO: 69 - protein, 15 aa, Artificial Sequence
  Ligand Variant
GGGCCGCAGGCTCCT

SEQ ID NO: 70 - protein, 15 aa, Artificial Sequence
  Ligand Variant
CTGGATAAATATGCG

SEQ ID NO: 71 - protein, 15 aa, Artificial Sequence
  Ligand Variant
CATCCCAAGTCTTAT

SEQ ID NO: 72 - protein, 15 aa, Artificial Sequence
  Ligand Variant
ACTCCTTCTTATTTG

SEQ ID NO: 73 - protein, 15 aa, Artificial Sequence
  Ligand Variant
AATCGCGAGAAGACT

SEQ ID NO: 74 - protein, 15 aa, Artificial Sequence
  Ligand Variant
AGTAAGCGTCAGCCG

SEQ ID NO: 75 - protein, 15 aa, Artificial Sequence
  Ligand Variant
CAGATTAAGCTTCTG

SEQ ID NO: 76 - protein, 15 aa, Artificial Sequence
  Ligand Variant
GGGACTAAGCATCGG

SEQ ID NO: 77 - protein, 15 aa, Artificial Sequence
  Ligand Variant
ATTAGCTTGCGGCCT

SEQ ID NO: 78 - protein, 15 aa, Artificial Sequence
  Ligand Variant
GGGACTGCGCGTCCT

SEQ ID NO: 79 - protein, 15 aa, Artificial Sequence
  Ligand Variant
GAGAATAAGCGTCCT

SEQ ID NO: 80 - protein, 5 aa, Artificial Sequence
  Ligand Variant
LDKYA

SEQ ID NO: 81 - protein, 15 aa, Artificial Sequence
  Ligand Variant
CTGGATAAATATGCT

SEQ ID NO: 82 - protein, 15 aa, Artificial Sequence
  Ligand Variant
TATGGTAATACTACG

SEQ ID NO: 83 - protein, 15 aa, Artificial Sequence
  Ligand Variant
GATCGGTCTCTTACG

SEQ ID NO: 84 - protein, 15 aa, Artificial Sequence
  Ligand Variant
TCGCATGGGCAGGAG

SEQ ID NO: 85 - protein, 15 aa, Artificial Sequence
  Ligand Variant
CATATTGCTGGTGCT

SEQ ID NO: 86 - protein, 15 aa, Artificial Sequence
  Ligand Variant
CCTAATCCTAGTCCG

SEQ ID NO: 87 - protein, 15 aa, Artificial Sequence
  Ligand Variant
GGTAAGTCGAGTAAG

SEQ ID NO: 88 - protein, 15 aa, Artificial Sequence
  Ligand Variant
CAGCCGCATTTGTCT

SEQ ID NO: 89 - protein, 15 aa, Artificial Sequence
  Ligand Variant
CCTCACGCGTCTCTT

SEQ ID NO: 90 - protein, 15 aa, Artificial Sequence
  Ligand Variant
CAGATGCAGTCGCGT

SEQ ID NO: 91 - protein, 10 aa, Artificial Sequence
  Ligand Variant
MYIEALDKYA

SEQ ID NO: 92 - protein, 5 aa, Artificial Sequence
  Ligand Variant
YRVKT

SEQ ID NO: 93 - protein, 5 aa, Artificial Sequence
  Ligand Variant
NSDSE

SEQ ID NO: 94 - protein, 5 aa, Artificial Sequence
  Ligand Variant
SHLVK

SEQ ID NO: 95 - protein, 7 aa, Artificial Sequence
  Ligand Variant
VVSHFND

SEQ ID NO: 96 - protein, 5 aa, Artificial Sequence
  Ligand Variant
NWVSE

SEQ ID NO: 97 - protein, 5 aa, Artificial Sequence
  Ligand Variant
NWRSE